(19) United States
(12) Patent Application Publication
(10) Pub. No.: US 2025/0179588 A1
(43) Pub. Date: Jun. 5, 2025

(54) PROCESSES, MACHINES, AND COMPOSITIONS RELATED TO ANALYZING NEOPLASMS SUCH AS CANCER

(71) Applicant: Flagship Pioneering Innovations VI, LLC, Cambridge, MA (US)

(72) Inventors: Kieran Ifan Chacko, Boston, MA (US); Sebastian Trousil, Brooklyn, NY (US); James Xin Sun, Needham Heights, MA (US)

(21) Appl. No.: 19/052,160

(22) Filed: Feb. 12, 2025

Related U.S. Application Data
(62) Division of application No. 17/561,077, filed on Dec. 23, 2021.
(60) Provisional application No. 63/133,004, filed on Dec. 31, 2020.

(51) Int. Cl. C12Q 1/6886; G16B 20/20

(52) U.S. Cl. CPC C12Q 1/6886; G16B 20/20; C12Q 2600/156

(57) ABSTRACT

Analyzing whether an individual has a neoplasm, such as an early-stage cancer, is based on detection of novel cancer-associated biomarkers in a biological sample. In one aspect, a process begins with a liquid biopsy from the individual, e.g., a blood sample, which is subjected to several sample processing steps resulting in a processed sample, such as a next generation sequencing (NGS) sample library. The processed sample contains cancer-informative cell-free DNA (“cfDNA”) from the liquid biopsy. The processed sample is subjected to DNA sequencing, to detect certain low-abundance cfDNA, for example by using a next generation DNA sequencer. Sequencer data output from the DNA sequencer is processed by a data processing system to provide a classification for the individual. For example, the data processing system can determine a likelihood of the individual having a neoplasm such as an early-stage cancerous tumor based on that individual's liquid biopsy.

### CROSS-REFERENCE TO RELATED APPLICATIONS

This application is a divisional application under 35 U.S.C. §§ 120 and 121 of U.S. patent application Ser. No. 17/561,077 filed Dec. 23, 2021, which claims the benefit of, and priority to, U.S. Provisional Patent Application No. 63/133,004 filed Dec. 31, 2020, the contents of each of which are incorporated herein by reference.

### BACKGROUND

Overall cancer mortality has decreased in the United States over the last 25 years, much of which can be attributed to greater awareness of cancer screening in the general population. The American Cancer Society (ACS) and the U.S. Preventive Services Task Force (USPSTF) provide cancer screening recommendations each year, with the aim of increasing the likelihood of benefits and limiting the harms from screening. These recommendations include, for example, mammography for detection of breast cancer, colonoscopy for detection of colon cancer, and pap smears for cervical cancer. However, most cancers detected in these tests are diagnosed only in later stages or after symptoms have manifested, making treatment more challenging.

### SUMMARY

This Summary introduces a selection of concepts in simplified form that are described further below in the Detailed Description. This Summary neither identifies key or essential features, nor limits the scope, of the claimed subject matter.

Analyzing whether an individual has a neoplasm, such as an early-stage cancer, is based on detection of novel cancer-associated biomarkers in a biological sample. In one aspect, a process begins with a liquid biopsy from the individual, e.g., a blood sample, which is subjected to several sample processing steps resulting in a processed sample, such as a next generation sequencing (NGS) sample library. The processed sample contains cancer-informative cell-free DNA (“cfDNA”) from the liquid biopsy. In some implementations, the processed sample is subjected to DNA sequencing, to detect certain low-abundance cfDNA, for example by using a next generation DNA sequencer. Sequencer data output from the DNA sequencer is processed by a data processing system to provide a classification for the individual. For example, the data processing system can determine a likelihood of the individual having a neoplasm such as an early-stage cancerous tumor based on that individual's liquid biopsy.

In one aspect, a system comprises computer storage and a processing system. The computer storage stores sequencer data corresponding to a processed sample, the processed sample including cell-free DNA fragments originating from a liquid biopsy of an individual and having been processed to enrich for cancer informative CGIs, the sequencer data comprising, for each sequenced cell-free DNA fragment corresponding to the cancer informative CGIs, a respective position on the genome for the cell-free DNA fragment and methylation information for the cell-free DNA fragment. The processing system accessing the computer storage to compute, based on the sequencer data, one or more metrics related to methylation for each of the cancer informative CGIs for the individual, and storing for the individual, for each of the cancer informative CGIs, the respective computed one or more metrics for the cancer informative CGI. The processing system accesses the metrics computed for the cancer informative CGIs for the individual and applying the accessed metrics to inputs of a computational model, the computational model providing an output indicating a likelihood of presence in the individual of a neoplasm. In some implementations, a DNA sequencer deep sequences the processed sample to a depth of greater than 200 times.

In one aspect, a process for highly selective identification of cell-free DNA of interest, includes sequencing cell-free DNA fragments in a biological sample under conditions sufficient to identify a methylation pattern occurring in the cell-free DNA fragments and computing one or more metrics related to the methylation patterns for cell-free DNA fragments in each of selected regions of interest of the genome. The computed one or more metrics for the selected regions are applied to a computational model, the computational model providing an output indicating a likelihood of presence of the cell-free DNA of interest. A combination of the one or more metrics, the selected regions, and the computational model provides highly selective identification of the cell-free DNA of interest.

In one aspect, a method includes processing a sample containing cell-free DNA fragments to obtain sequencer data about methylation occurring in the cell-free DNA fragments in cancer informative CGIs in the sample and computing one or more metrics for each of the cancer informative CGIs, wherein each metric for a cancer informative CGI comprises a different function of the sequencer data corresponding to the cell-free DNA fragments in the respective cancer informative CGI. The computed one or more metrics for the cancer informative CGIs are applied to a computational model, the computational model providing an output indicating a likelihood of presence in the individual of a neoplasm.

In one aspect, a method of processing a DNA-containing sample from a subject, includes processing the sample to enrich cell-free DNA originating from cancer informative CGIs and using an analytical platform to detect a methylation pattern in the cell-free DNA in the sample from the cancer informative CGIs, wherein the cancer informative CGIs are selected from a group consisting of a ranked set of candidate CGIs.

In one aspect, a method of processing a DNA-containing sample from a subject, using at least one cancer informative CGI detected in the sample having a methylation pattern in combination with at least one metric related to the methylation pattern of the at least one cancer informative CGI, detects or monitors for the presence of cancer or a precancerous state or condition or to predict the likelihood of cancer or a precancerous state or condition with a high sensitivity and high specificity.

In another aspect, a method of processing a DNA-containing sample from a subject, using a combination of one or more cancer informative CGIs, one or more metrics related to methylation patterns of the cancer informative CGIs, and a computational model, predicts a likelihood of presence of an early-stage neoplasm with a high sensitivity and a high specificity.

In another aspect, a method of processing a DNA-containing sample from a subject, using an analytical platform, detects a desired methylation pattern on at least one cancer informative CGI, as listed herein, to detect or monitor for the presence of cancer or a precancerous state or condition or to predict the likelihood of cancer or a precancerous state or condition with a high sensitivity and a high specificity.

In any of the foregoing, the cell-free DNA can be suitable to detect or monitor the presence of early-stage cancer or a precancerous state/condition or to predict the likelihood of cancer or a precancerous state/condition.

In any of the foregoing, the one or more metrics can include one or more metrics selected from the group consisting of the list of metrics in FIG. 6-4.

In any of the foregoing, the sample can include plasma obtained from an asymptomatic individual.

In any of the foregoing, a combination of one or more metrics and selected cancer informative CGIs can provides the computational model with a sensitivity and specificity suitable to predict a likelihood of presence in the individual of a neoplasm.

In any of the foregoing the neoplasm can be an early-stage cancerous solid tumor.

In any of the foregoing the selected cancer informative CGIs can include those having a statistical metric above a threshold based on a training set and one or more metrics computed for samples in the training set.

In any of the foregoing, the one or more metrics can include one or more metrics selected from among metrics listed in FIG. 6-4.

In any of the foregoing, processing the sample can include detecting or monitoring for the presence of cancer or a precancerous state/condition or to predict the likelihood of cancer or a precancerous state/condition with a sensitivity greater than Y and/or a specificity greater than X based on cfDNA found in the sample.

In any of the foregoing, a combination of the one or more metrics and the selected cancer informative CGIs can provide the computational model with a sensitivity of >70% at a specificity of greater than about 95%.

In any of the foregoing, the cancer informative CGIs can be selected from a group consisting of a top X of a set of ranked CGIs, where X is a positive integer, based on a process of ranking CGIs using sequencer data derived for a training set. The CGIs listed in TABLE A can be ranked by using sequencer data derived for a training set, and the ranked CGIs can be used to select as set of cancer informative CGIs.

In any of the foregoing, the cancer informative CGIs can include those having a statistical metric, e.g., a p-value, above a threshold, based on sequencer data for a training set and one or more metrics related to methylation in CGIs and computed for the training set. These one or more metrics can be selected from among metrics listed in FIG. 6-4. Any one or more of the following selections of metrics can be used. The selected metric is an averaging metric. The selected metric is a haplotype metric. The selected metric is a proportion metric. The selected metric is a transition metric. The CGIs listed in TABLE A can be processed by applying such metrics to sequencer data derived for a training set to provide ranked CGIs from which a selection can be made.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIG. 1 is a data flow diagram of an example system for determining whether an individual has a likelihood of having a neoplasm, such as an early-stage cancerous tumor.

FIG. 2 is a data flow diagram of an example system for obtaining sequencer data from liquid biopsies.

FIG. 3 is an illustrative example of sequencer data for a single CGI.

FIG. 4 is a data flow diagram of an example system for processing sequencer data.

FIG. 5 is a block diagram of an example general purpose computer.

FIGS. 6-1 through 6-4 illustrate different example metrics.

FIG. 7 is a flow chart of an example process for sample preparation.

In the drawings, in the data flow diagrams, a parallelogram indicates an object that is an input to a system that manipulates the object or an output of such a system, whereas a rectangle indicates the system that manipulates that object.

### DETAILED DESCRIPTION

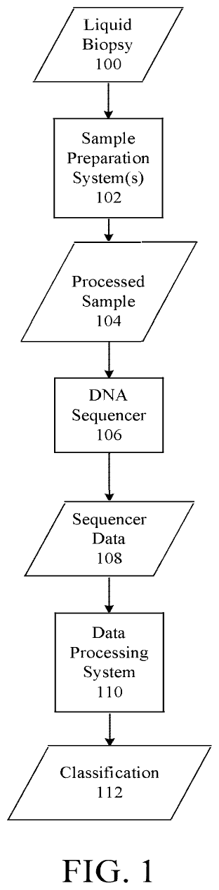

Referring now to the data flow diagram of FIG. 1, an example system for determining whether an individual has a likelihood of having a neoplasm, such as an early-stage cancerous tumor, will be described. A liquid biopsy 100 from an individual is obtained. The liquid biopsy is subjected to several sample processing steps, represented by sample preparation system(s) 102, examples of which are described in more detail below, to provide a processed sample 104, such as a next generation sequencing (NGS) sample library. The processed sample 104 is subjected to DNA sequencing by a DNA sequencer 106. The DNA sequencer 106 outputs what is referred to herein as the “sequencer data” 108, also described in more detail below. The sequencer data 108 output from the DNA sequencer 106 is processed by data processing system 110 to provide a classification 112 for the individual. For example, the data processing system 110 can determine a likelihood of the individual having a neoplasm such as an early-stage cancerous tumor based on that individual's liquid biopsy. Details of an example implementation of such a data processing system 110 are provided in more detail below.

Detecting a neoplasm, such as early-stage cancer, in an individual based on a liquid biopsy involves addressing several challenging technical problems in both biology and machine computation. If an individual has a neoplasm, e.g., a cancerous tumor, then cell free DNA (cfDNA) from cells from the tumor will appear in the blood stream. However, cfDNA from healthy cells also is present in the blood stream. Also, DNA is present in the blood stream from other sources, such as white blood cells. Thus, each DNA fragment found in the liquid biopsy can originate from a variety of possible sources. Also, DNA fragments originating from a neoplasm will occur in a low quantity in the blood if the neoplasm is only in an early stage of development, such as a very early-stage cancer.

The DNA fragments found in the liquid biopsy also can be from anywhere in the genome. Certain portions of a genome comprise regions with a high frequency of CpG sites. The term “CpG site” means a location in DNA where cytosine and guanine are separated by only one phosphate group. Such regions are referred to as CG islands or CGIs. It has been found that certain CGIs and certain features of certain CGIs in tumor cells tend to be different from the same CGIs or features of the CGIs in healthy cells. Herein, such CGIs and features of the genome are referred to herein as “cancer informative CGIs”, which is defined and described in more detail below. These cancer informative CGIs tend to have methylation patterns in tumor cells that are different from the methylation patterns in healthy cells. DNA fragments from other CGIs may not express such differences.

Thus, some of the challenges in processing liquid biopsies include, but are not limited to, a low quantity of cfDNA from a neoplasm, if the neoplasm is in an early stage, significant “noise” due to the presence of a high volume of cfDNA from healthy or non-tumorous cells, as well as DNA fragments and whole genomes originating from white blood cells, and significant noise due to cfDNA, from either tumor or non-tumor cells, which is not informative about the presence of a neoplasm or cancer.

In addition, sequencing the DNA fragments from such samples produces a large volume of sequencer data. From a signal processing point of view this large volume of data includes a lot of noise and the desired signal, i.e., any data informative of the presence of a neoplasm, is a comparatively small signal.

To address these challenges, implementations of a system, or its components, can include one or more of the following. First, by processing sequencer data from a training set of samples for which a classification, e.g., cancer or tumor yes/no, is known, a set of cancer informative CGIs can be identified and selected from among a larger set of CGIs. Second, metrics related to the methylation patterns within these CGIs can be computed. Such metrics can be used to assist in identifying and selecting cancer informative CGIs. Third, given a set of cancer informative CGIs, samples can be processed to enrich for the cfDNA fragments containing the cancer informative CGIs. Fourth, sample libraries can be deep sequenced, using next generation sequencers, on the order of several hundred times, so that there is, on average, a large number of cfDNA fragments sequenced for the cancer informative CGIs, thus increasing the likelihood of sequencing cfDNA fragments originating from a neoplasm. Fifth, using the sequencer data for the training set, a computational model can be built that can predict likelihood of presence of neoplasm in other individuals based on liquid biopsies from those individuals. In some implementations, such a computational model can be built using various machine learning techniques and classification models. Specifically, a set of features can be computed from sequencer data for the training set, where the features are the combination of one or more metrics for selected cancer informative CGIs, and a model can be trained such that the trained computational model has a sensitivity and specificity suitable to predict a likelihood of presence in the individual of a neoplasm such as an early-stage cancerous solid tumor. Finally, the computational model can be used to process the sequencer data originating from liquid biopsies of other individuals to screen for the likelihood of presence of a neoplasm.

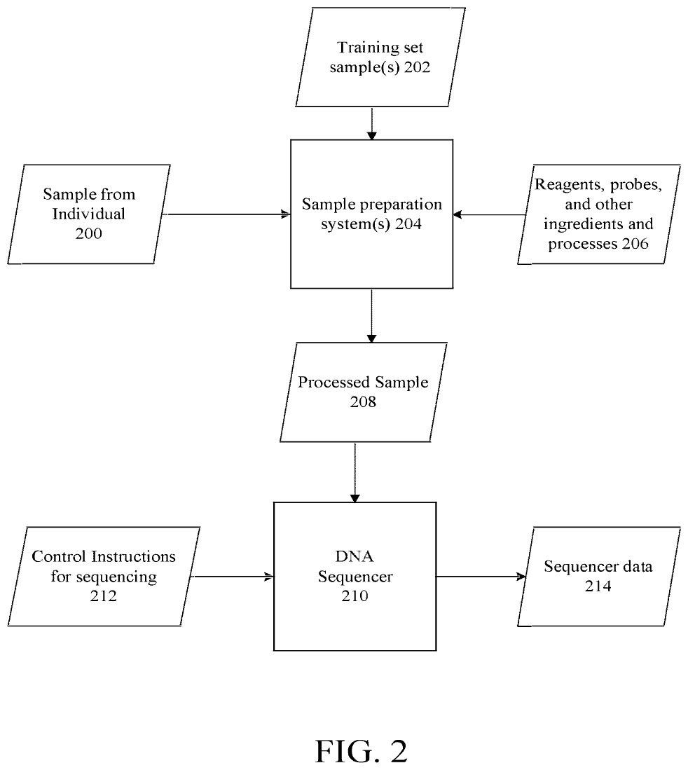

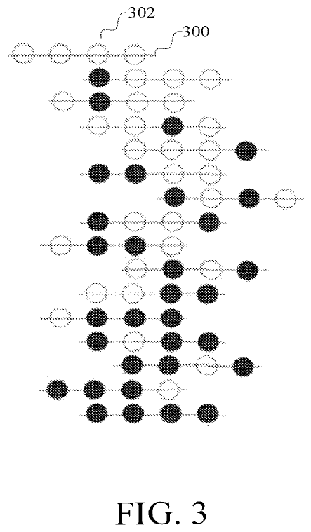

Referring now to the data flow diagram of FIG. 2, an example system for obtaining sequencer data from liquid biopsies will now be described. This diagram illustrates an example system which processes a set of liquid biopsies, for what is called herein a training set, to obtain sequencer data for the training set, and which processes one or more liquid biopsies, for one or more individuals for whom screening is being performed, to obtain sequencer data for the one or more liquid biopsies. As noted above, the training set comprises a set of samples for which a classification, e.g., cancer or tumor yes/no, is known. Other information about the classification may be known such as a tissue of origin or the part of the body affected by the neoplasm, a stage of development of a cancer or other kind of tumor, a type of cancer or other kind of tumor.

Samples from individual(s) 200, or sample(s) from the training set 202, are input to the sample processing system(s) 204. Such systems 204 generally use reagents, probes, and other ingredients and processes 206 to process a sample 200 or 202 to obtain a processed sample 208 for sequencing. Generally, but not necessarily, the sample processing system(s) used to process both kinds of samples 200 and 202 are the same.

The processed sample 208, such as an NGS sample library, is placed in the DNA sequencer 210. The DNA sequencer 210 sequences the prepared sample based on control instructions 212, such as instructions about the depth of sequencing to be performed. The output of the DNA sequencer 210 is the sequencer data 214.

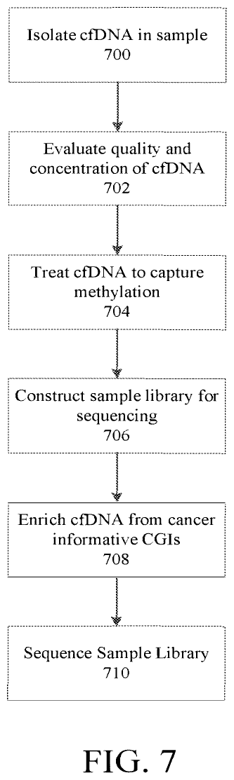

Further details for example implementations of the components illustrated FIG. 2 will now be described in connection with FIG. 7. FIG. 7 is only an example process for preparing a sample, especially a blood sample. In some cases, it is possible to skip step 702. Also, the various steps 704, 706, and 708 may be performed in different orders. Also, different kinds of preparation are performed for different kinds of sequencing or analytical platforms. The example in FIG. 7 describes creating a processed sample which is an NGS sample library to be used with an Illumina NovaSeq sequencer.

In some respects, a liquid biopsy (i.e., blood sample) or a tissue biopsy (i.e., whole cells from a tissue) is obtained form an individual. The biopsy is treated in order to isolate (700) the cfDNA. Liquid biopsies are preferred, as solid tissue biopsies are expensive and invasive, making them less than ideal for patients who are older or very young, or for cancers that are not readily accessible, such as most lung cancers. CfDNA derived from blood or other biological liquid represents a more accessible material from which DNA can be obtained for next-generation sequencing (NGS) testing. The amount of cfDNA yields may vary widely between patients, cancer type, and cancer stage but are nearly always low, averaging approximately 20-30 ng/10 cc blood draw. The cfDNA is characterized by a uniform fragmentation size of about 100-300 base pairs (bps) indicating its being derived from nucleosome-protected DNA resulting from in vivo cleavage occurring as cells undergo apoptosis and/or necrosis. Therefore, cfDNA yield is related to the type of cancer. Cancers associated with a high degree of apoptosis will have the larger yields.

Collection of blood for cfDNA studies uses some procedures unique to such studies. cfDNA is usually purified from the plasma fraction which is devoid of white blood cells. This is done to prevent genomic DNA (gDNA) contamination resulting from white blood cell lysis. Genomic DNA contamination is detected as high molecular weight DNA. This sort of contamination will dilute out the tumor cfDNA, reducing the ability to detect rare variants. Techniques are available that preferentially remove high-molecular weight DNA from cfDNA contaminated with gDNA and have used it on both new samples and archival plasma samples. Techniques for enriching cfDNA are described, for example, in Gilson, Enrichment and Analysis of ctDNA, (2020), Recent Results Cancer Res., 215:181-211. [1]

Briefly, the cfDNA obtained from a liquid biopsy sample is processed to isolate the cfDNA (700) and then evaluate (702) its quality and concentration. Instruments for assessing the quality of the cfDNA, such as the TapeStation System from Agilent Technologies (Santa Clara, CA) can be used. Concentrating low-abundance cfDNA can be accomplished, for example using a Qubit Fluorometer from Thermofisher Scientific (Waltham, MA).

The cfDNA may be treated (704) to capture the methylation modifications, e.g., using bisulfite conversion. Bisulfite conversion enables highly efficient conversion of unmethylated cytosines to uracils of DNA from samples such as whole blood or plasma, cultured cells, tissue samples, genomic DNA, and formalin-fixed, paraffin-embedded (FFPE) tissues. Bisulfite conversion can be performed using commercially available technologies, such as Zymo Gold available from Zymo Research (Irvine, CA) or EpiTect Fast available from Qiagen (Germantown, MD). Other techniques include but are not limited to enzymatic methods.

A sample library is then constructed (706), by adding one or more “adapters” to the DNA fragments. The adapter(s) are compatible with the DNA sequencer to be used and enable the DNA sequencer to detect the DNA fragment for sequencing. Library construction may be accomplished, for example using commercially available technologies, such as the Accel-NGS Methyl-Seq DNA Library prep technology from Swift Biosciences (Ann Arbor, MI). Processed samples from multiple individuals can be combined by adding unique indexes to each of the cfDNA fragments, to allow pooled sequencing.

Sample multiplexing, also known as multiplex sequencing, allows large numbers of libraries to be pooled and sequenced simultaneously during a single run on a sequencing instrument. Sample multiplexing is useful when targeting specific genomic regions or working with smaller genomes. Pooling samples exponentially increases the number of samples analyzed in a single run, without drastically increasing cost or time.

With multiplex sequencing, individual “barcode” sequences may be added to each DNA fragment during NGS library preparation so that each read can be identified and sorted before the final data analysis.

Pooling of patient samples increases the effectiveness of NGS, which involves sequencing a large number of genomes at high coverage. Sequencing DNA from pools of individuals has benefits such as needing less DNA from each single individual and reducing overall work and time of sequencing experiments. Anand et al., “Next Generation Sequencing of Pooled Samples: Guideline for Variants' Filtering”. Sci Rep 6, 33735 (2016) [3]; Bilder and Tebbs, “Pooled testing procedures for screening high-volume clinical specimens in heterogeneous populations” Stat Med. 2012 Nov. 30; 31(27): 3261-3268 [4].

Methods for preparing pooled libraries for NGS are well-established, and include techniques such as barcoding and combinatorial pooled sequencing. Shokralla et al., “Next-generation DNA barcoding: using next-generation sequencing to enhance and accelerate DNA barcode capture from single specimens”, Mol. Ecol Resources (2014)14, 892-901 [5]; Cao and Sun, “Combinatorial pooled sequencing: experiment design and decoding”, Quantitative Biology 2016, 4(1): 36-46 [6].

The cfDNA in the constructed sample library then may be enriched (708) for selected cancer informative CGIs. This step may be accomplished using hybrid capture probe sets that enable targeting of selected genomic regions, followed by PCR amplification. Examples of such hybrid capture probe sets include the KAPA HyperPrep Kit and SeqCAP Epi Enrichment System from Roche Diagnostics (Pleasanton, CA). The selected genomic regions may be those cancer informative CGIs selected and used for training a computational model, or selected and used in a trained computational model, as described herein.

The enriched sample library containing the cfDNA then is ready for sequencing (710), which may be accomplished, for example, using a commercially available sequencer such as the Illumina NovaSeq, the output of which is a FASTQ format data file.

Such sequencing is commonly referred to as “deep sequencing” or “next generation sequencing”. The sample library is sequenced to a “depth”, set at the time of sequencing of the sample library. The sample library is sequenced to a depth of “X”, a positive integer, where X is determined based on the desired average coverage of the cancer informative CGIs in the sample library.

After a sample library has been constructed from a liquid biopsy, the sample library contains DNA fragments from cancer informative CGIs but in an unknown distribution. Not all cancer informative CGIs will have corresponding DNA fragments in the sample library; not all DNA fragments will be from cancer informative CGIs. A DNA fragment may be only a portion of a cancer informative CGI, and may include portions not within the cancer informative CGI.

Depth of sequencing refers to the number of unique sequenced DNA molecules for a given genomic position within a cancer informative CGI. A higher depth of sequencing yields, on average, more sequenced unique DNA fragments from the cancer informative CGIs. Due to the expected very low proportion of circulating tumor DNA (ctDNA) amongst all cfDNA in a patient liquid biopsy sample, particularly for neoplasms such as early-stage cancer and precancerous states, deep sequencing is used to increase the probability of sequencing the rare ctDNA fragments. Additionally, deep sequencing is used to compute the methylation values more precisely from DNA fragments within the cancer informative CGIs.

We have discovered that a depth of sequencing greater than 200 unique reads per position within a cancer informative CGI (>200×) is preferable. Any depth of sequencing above 200× can be used depending on the number of cancer informative CGIs, type of cancer to be detected, stage of cancer to be detected, quality of the biopsy, quality of the sample processing, and level of enrichment performed in the sample processing. The depth of sequencing can be greater than 250×, greater than 300×, greater than 350×, greater than 400×, greater than 450×, greater than 500×, greater than 550×, greater than 600×, greater than 650×, greater than 700×, greater than 750×.

Referring now to FIG. 3, an illustrative example of a portion of sequencer data will now be described.

In FIG. 3, sequencer data, for the purposes described herein, generally includes, for each DNA fragment, data indicating a location in the genome for the fragment, and, for each CPG within the DNA fragment, data indicating whether that CPG is methylated. However, when output by a DNA sequencer into a FASTQ format data file, the raw data can be many gigabytes, e.g., 20 to 30 gigabytes, of data per individual.

Conceptually, as illustrated in FIG. 3, the sequencer data includes data for sequenced DNA fragments of cancer informative CGIs. In FIG. 3, a row, e.g., row 300, represents a sequenced DNA fragment. Thus, in FIG. 3, data for sixteen DNA fragments is shown. In FIG. 3, each circle corresponds to a CPG. Whether the circle is black or white is indicative of whether the sequencer detected that CPG as methylated (black) or not (white). In FIG. 3, the data for the DNA fragments within a CGI are illustrated as aligned by the respective locations of the DNA fragments in a genome. Thus, column 302 corresponds to the same CPG in the genome. Note that, in this illustration, four DNA fragments do not have methylation information for this CPG.

Using the location information for each DNA fragment from the sequencer data, data corresponding to the DNA fragments can be grouped by cancer informative CGIs in which the DNA fragments are found. Thus, the example in FIG. 3 can be considered to illustrate how the data for DNA fragments in one cancer informative CGI can be collected and aligned. Such segmentation of the sequencer data for each patient into data by cancer informative CGI can be performed for a full set of cancer informative CGIs, resulting in a respective subset of the sequencer data for each cancer informative CGI.

The example implementation is described herein in the context of analyzing cfDNA based on DNA sequencing. Other DNA analysis methods (e.g., PCR) can be used to obtain similar information about methylation state of cfDNA fragments, such as shown in FIG. 3. The term “sequencer data” herein is intended to include any data that provides information about methylation of CPGs in selected cancer informative CGIs regardless of the origin or equipment used to obtain that information.

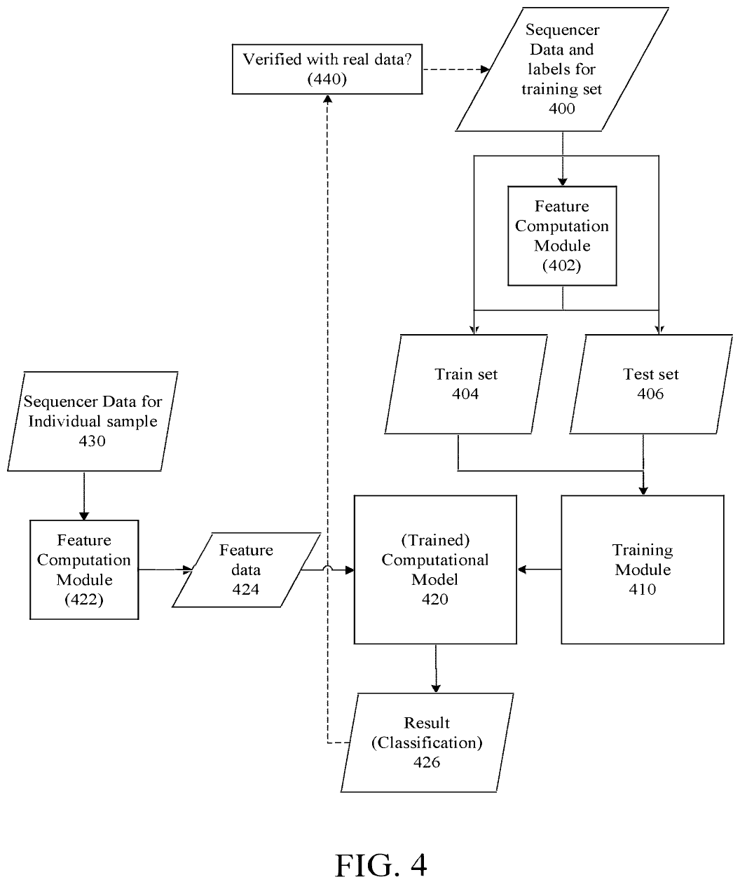

Referring now to FIG. 4, a data flow diagram of an example system which processes sequencer data will now be described.

This diagram illustrates the processes of processing the sequencer data for what is called the training set (400), as well as for processing sequencer data for a liquid biopsy for an individual (430) for whom screening is being performed.

As noted above, the training set comprises a set of samples (e.g., liquid biopsies) for which respective classifications for each sample are known. Examples of known classifications for the samples, typically called “labels”, include information such as a cancer or tumor yes/no, type of cancer, stage of cancer, a tissue of origin or the part of the body affected by a neoplasm, or type of neoplasm. Data called “features” are derived from the sequencer data obtained from the training set. These features are used to build or train a computational model that can classify unknown samples using features computed from the sequencer data for those samples. The sequencer data for the liquid biopsy for the individual for whom screening is being performed is an input, from which features are computed and input into this trained computational model. The trained computational model provides an output indicative of the likelihood of presence of a neoplasm such as an early-stage cancer in the individual. A training set generally includes samples both with and without a neoplasm such as cancer, from multiple stages including early stages of development, and from multiple types of tumors or cancers.

As illustrated in FIG. 4, the sequencer data and labels for the training set (400) are processed to compute features to be used by a computational model 420. FIG. 4 illustrates a “feature computation module” 402 that performs such processing. The combination of computed features and the labels are used by a training module 410 to train the computational module 420. Typically, such training is performed by dividing the sets of features computed for different samples in the training set into a train set 404 and a test set 406, and continually adjusting parameters of the trained computational module using the train set while minimizing errors in classifying the test set.

The feature computation module 402, for which example implementations are described in more detail below, computes one or more metrics related to the methylation for each of the cancer informative CGIs selected to build the computational model. The specific metrics used and cancer informative CGIs selected can depend on the training set and desired classification. Example selections are provided in more detail below. The selected metric(s) for the selected cancer informative CGIs are computed for the training set to provide the train set and the test set. Generally, the combination of the one or more metrics and the cancer informative CGIs are selected such that, given the training set, the trained computational model has a sensitivity and specificity suitable to predict a likelihood of presence in the individual of a neoplasm, such as an early-stage cancerous tumor. Such selection is described in more detail below.

Given a trained model 420, that model can be used to process features computed from sequencer data for an individual, unknown or unclassified, sample 430. A feature computation module 422, which performs similar computations as module 402, processes the sequencer data for an individual to compute the one or more metrics for the selected cancer informative CGIs (used in the trained computational model 420) based the sequencer data 430 to provide feature data 424. This feature data 424 is input to the trained computational model 420, which provides a result, a classification 426, for that individual sample. Note that if this classification 426 is verified as indicated at 440, the data for the sample could be added to the data for the training set, and can be used to update the trained computational model.

Typically, to train a computational model, a training data set is used. The training data set includes data for examples for which a prediction or label or other outcome is known. To predict the likelihood of presence of a neoplasm, the training data set includes data for examples where there is no tumor or cancer, and where there is a tumor or cancer. As an example, a training set of 452 liquid biopsies (blood) was obtained, of which 396 were known to be from individuals with cancer across different stages (I, II, and III) and 56 were known to be from individuals who were cancer free. Generally, the more samples obtained the better, with a distribution of patients across different types of neoplasms to be detected and across different stages of development to be detected. The liquid biopsies from such individuals provide a training set, which is processed as described above, and then sequenced to produce corresponding sequencer data for the training set. The sequencer data is then processed to provide a set of computed features for each sample.

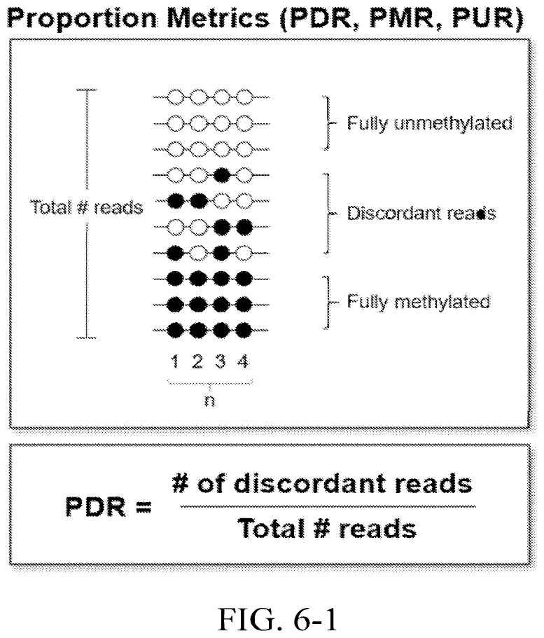

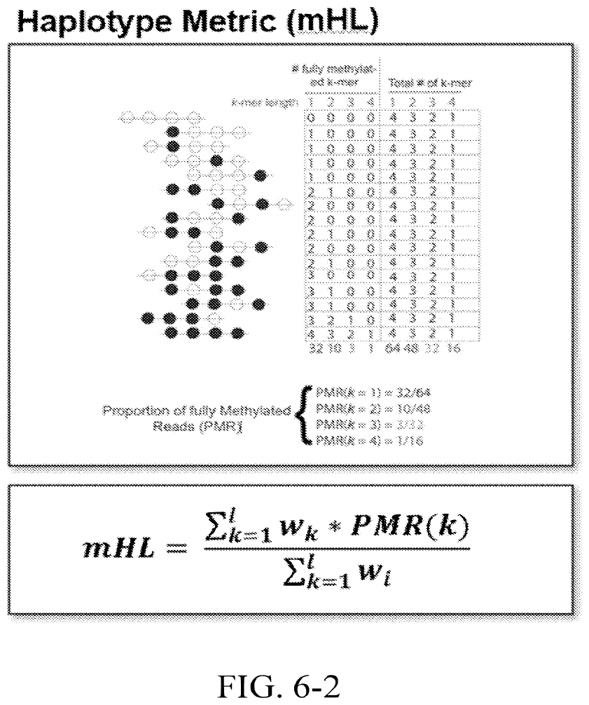

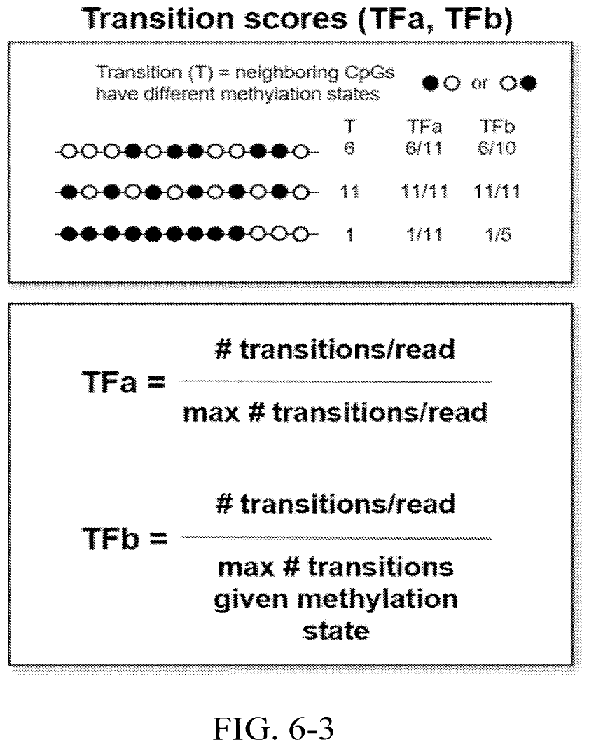

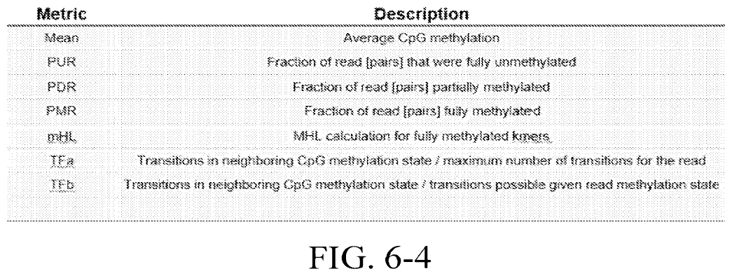

As mentioned above, a feature computation module computes one or more metrics for selected cancer informative CGIs based the sequencer data related to that CGI. The following are some example metrics that can be computed for a CGI. These example metrics are summarized in FIG. 6-4. A variety of other metrics can be used.

Averaging metrics for a CGI are related to the mean methylation of the DNA fragments in the CGI. In the table in FIG. 6-4, one “mean” metrics is listed, but represents any of many possible forms of such a calculation, such as a classical average, or an average normalized for coverage, or an average modified for density, as examples.

Proportion metrics for a CGI are related to the relative proportion(s) of the DNA fragments in the CGI which are fully methylated, fully unmethylated and discordant (partially methylated and partially unmethylated). See FIG. 6-1. In the example of FIG. 6-1, in several DNA fragments, all relevant CPGs are methylated, and thus these three are called fully methylated. There are four which are partially methylated in the relevant CPGs, and three which are fully unmethylated. The “PDR”, or proportion of discordant reads, is the ratio of the number of partially methylated fragments to the total number of fragments (in this illustrative example, ten). The “PUR”, or the proportion of unmethylated reads, is the ratio of the number of fully unmethylated fragments to the total number of fragments. The “PMR”, or the proportion of methylated reads, is the ratio of the number of fully methylated fragments to the total number of fragments. Note also that a proportion metric can be computed for any selected number of any selected CPGs within a CGI. Such metrics are described in more detail, for example, in U.S. Patent Publication 2020/0109456A1 [2] and Landau et al., “Locally disordered methylation forms the basis of intra-tumor methylome variation in chronic lymphocytic leukemia”, in Cancer Cell, 2014 Dec. 8, 26(6):813-825 [7].

Another kind of metric is called herein a haplotype metric, an example of which is called “mHL” herein. This metric uses the PMR metric from above, but as a function of a positive integer number k of consecutive fully methylated reads, as illustrated in FIG. 6-2. Different metrics with different values of K can be computed. Such a metric is described in more detail, for example, in Guo S., et al., “Identification of methylation haplotype blocks aids in deconvolution of heterogeneous tissue samples and tumor tissue-of-origin mapping from plasma DNA”, Nat. Genet. 2017 April; 49(4):635-642 [8].

Another type of metric is called herein a transition metric. See FIG. 6-3. For a given DNA fragment, the number of transitions between unmethylated and methylated is computed. The ratio of this number to another number, such as the total number of transitions (TFa), or a maximum number of transitions in methylation state (TFb) is determined. An average of transitions scores for all DNA fragments in a selected CGI can be computed.

A variety of other metrics also can be developed and used, and the invention is not limited to the set described herein. Further, as noted below, a different respective set of one or more metrics can be selected to be computed for each selected cancer informative CGI. The selection of the cancer informative CGIs can be influenced by both the selected metrics and the training set for which there is sequencer data to which the metrics are applied.

The combination of metrics and cancer informative CGIs for which they are computed are used to create a set of features based on the sequencer data from a training set, or for a sample to be screened.

Thus, referring again to FIG. 3 and sequencer data obtained for a sample, sequencer data is first grouped by individual, and then, for each individual, the data for DNA fragments are grouped by CGI and aligned by CPG. The data representing the methylation information for each DNA fragment within the CGI is reduced by computing one or more of the metrics for the CGI. Thus, the raw sequencer data is reduced for an individual to a collection of the metrics computed for each of the selected cancer informative CGIs. Such data can be represented in a table where each row is a CGI and each column includes a metric computed for that CGI.

When using sequencer data from a training set as described above to create a predictive model or classifier, there are several technical problems to be addressed in the realm of machine computation. Among these problems is the amount of data to be processed. With a large number of reads of a large number of DNA fragments over a large number of CGIs from a sample, the amount of data received from the DNA sequencer is several gigabytes of data. Even when the amount of data is reduced by computing metrics related to methylation for certain CGIs, if the numbers of possible metrics and possible CGIs are high, then a large number of features (a pair of a metric and the CGI for which the metric is computed) per sample will be obtained. However, there may be a relatively small number (hundreds or thousands) of samples in the training set, as it is difficult to obtain a good training set of samples known to be related to neoplasms such as early-stage cancers.

If such a data set is used to train a computational model, several problems can arise. In some instances, features that are correlated but contain valuable information to enable a classification can be dropped from use, due to the nature of the optimization performed by different training processes for different models. The loss of information can result in decreased specificity and sensitivity of the trained model. In some instances, the small number of samples in the training set can result in a trained model that is “overfit”, meaning that the model does very well so long as any new sample to be screened is similar to those in the training set, but otherwise, the model performs poorly (e.g., has decreased specificity and sensitivity).

To address such problems, a variety of techniques can be used, whether alone or in combination. For example, the number of cancer informative CGIs selected for consideration can be limited, in a manner described in more detail below. As another example, the number of metrics computed for each CGI can be limited, an example of which also is described in more detail below. This process typically is called feature selection. Its results can depend on the size and quality of the training set.

Other techniques that can be used relate to the selection and training of the model. Some model types, such as a random forest or other form of classification or decision tree, are less susceptible to overfitting given a small training set. Another kind of model involves creating an ensemble of different models, where the different models are trained with different subsets of features from the training set. These different subsets of features are selected so that highly correlated features are in different subsets. Yet other model types typically are called “deep learning” models which, in a sense, discover features in the data from the training set.

Such computational models are known by a variety of names, including, but not limited to, classifiers, decision trees, random forests, classification and regression trees, clustering algorithms, predictive models, neural networks, genetic algorithms, deep learning algorithms, convolutional neural networks, artificial intelligence systems, machine learning algorithms, Bayesian models, expert rules, support vector machines, conditional random fields, logistic regression, maximum entropy, among others. In general, such models receive a vector or n-dimensional matrix of features as an input, and provide an output such as a classification, prediction, or other value. The computational model used for classification may or may not be a computational model that is trained by a training set. For example, the computational model can be a simplified set of computations performed on features computed for an individual, where that set of computations is based on insights obtained by analyzing data from a training set. Typically, the computational model computes a function of the features, which may be a linear or non-linear function, to produce an output where the output is indicative of the resulting classification.

The output of the computational model is a form of prediction, indicating a likelihood that the individual from whom a sample was obtained has a neoplasm, such as an early-stage cancerous tumor or other solid tumor present in the body. This prediction can be in the form of a probability between zero and one, or a binary output, such as a yes or no answer, or a score (which may be compared to one or more thresholds), or other format. The output can be accompanied by additional information indicating, for example, a level of confidence in the prediction. The output typically depends on the form of the computational model used.

In some implementations that output indicates the likelihood of the presence of a neoplasm such as cancer, without indicating a type of tumor or cancer, i.e., the affected tissue. In some implementations, the output of the model can indicate a type of cancer. In some implementations, the output of the model can indicate a type of cancer, and then one or more additional models can be applied to the data to indicate a type of cancer. In some implementations, a separate model for each type of cancer can be used, and an ensembling process can process the outputs of the separate models.

An overview of a selection of cancer informative CGIs will now be provided. There are approximately 26000 commonly recognized CGIs in the human genome. Not all of them are cancer informative. For the purposes of an example implementation, a set of potentially cancer informative CGIs is used as a starting point. This example is based on the CGIs identified in U.S. Patent Publication 2020/0109456A1 [2], which is hereby incorporated by reference, specifically the “Table I” of CGIs listed in that published patent application.

For the purposes of simplifying processing, each cancer informative CGI can be a “CGI identifier” or reference number to allow referencing CGIs during data processing by their respective unique CGI identifiers. The accompanying table entitled “Table A—Listing of CGIs” (also herein, “TABLE A”) lists, for each CGI, its respective location in the human genome.

One of the challenges to be addressed is, given a training set if known samples, how to select which cancer informative CGIs and which metrics to compute for the selected cancer informative CGI, so as to have a set of features to use to make a predictive computational model. Generally, the combination of the one or more metrics and the cancer informative CGIs are selected such that, given the training set, the trained computational model has a sensitivity and specificity suitable to predict a likelihood of presence in the individual of a neoplasm, such as an early-stage cancerous solid tumor. Sensitivity is the true positive rate, reported as a proportion of correctly identified positives; Specificity is the true negative rate reported as a proportion of correctly identified negatives.

One way to improve the sensitivity and specificity is to reduce the number of features (i.e., pairs of CGIs and respective metrics) used to train a model from an initial larger set of candidate features, such as a large number of CGIs and metrics. As one example implementation, given the example training set described above, a p-value for each metric was computed for each candidate CGI in TABLE A. The best p-value across metrics was selected for each CGI, and the CGIs were ranked by their respective best p-values. In null hypothesis significance testing, the p-value is the probability of obtaining test results at least as extreme as the results actually observed, under the assumption that the null hypothesis is correct. A very small p-value means that such an extreme observed outcome would be very unlikely under the null hypothesis. Thus, when evaluating candidate CGIs to select a set of cancer informative CGIs to use to build a computational model, p-values can be computed for each respective metric computed for each CGI, and the best (i.e., lowest) value can be selected for each CGI, and the CGIs can be ranked. A threshold can be applied to this ranking based on the p-value. Such a threshold can be defined by a formula of “X×10^{-Y}” (often written as “X E-Y”, e.g., “9.0 E-8”, for 9.0 times 10⁻⁸) wherein X is any number greater than zero and less than 10, and Y is a positive integer number. Example values of Y that can be useful as a threshold for the p-value is selected cancer informative CGIs are 10, 11, 12, 13, 14, 15, or 16.

This process typically is iterative. A set of candidate CGIs and set of candidate metrics is initially selected. Given sequencer data for a training set, the candidate metrics are computed for the candidate CGIs. The candidate CGIs can be ranked using a statistical metric applied to the computed metrics and how well they classify samples from the training set. A selection of CGIs can be made from the ranked candidate CGIs, and a computational model can be built and tested. The statistical metrics, metrics applied to sequencer data, and selections of ranked CGIs can be iteratively evaluated until the selections result in a model with suitable sensitivity and specificity.

Given the CGIs in TABLE A, a subset of the CGIs can be selected as the selected cancer informative CGIs to be used to build a computational model using the sequencer data from the training set. That computational model can be applied to sequencer data for liquid biopsies to be screened. The cancer informative CGIs so selected and used by the computational model also can inform the sample preparation process and the DNA sequencing process, so that only the selected cancer informative CGIs are used for hybrid capture and PCR amplification to prepare a processed sample.

It should be understood that any specific ranking of CGIs would be dependent on the metrics computed for those CGIs based on sequencer data for any available training set. Different training sets will likely result in different rankings. Further, the ranking can be performed using any other statistical metric that is useful for identifying those metrics and CGIs that have a stronger ability to determine the classification of samples. Additionally, given a ranking of CGIs by a statistical metric applied to the features computed for a given training set, a selection of the cancer informative CGIs to be used for training can be performed in many ways.

For example, in one implementation, 359 CGIs from TABLE A were used to train a random forest model, resulting in a model with a sensitivity of about 74% was obtained for the trained model at a specificity of about 95%.

Examples selections of CGIs from TABLE A include the following: A first number N of CGIs selected from a subset of the CGIs where the subset of CGIs is a second number M of the CGIs listed in TABLE A, where N<M and N and M are positive integers. Example values of N are 1, 2, 3, 4, 5, 6, 7, 8, 9, 10, 11, 12, 13, 14, 15, 16, . . . , 400, in increments of 1, so long as N<M, and example values of M are 10, 20, 30, 40, 50, . . . , 1590, in increments of 10. The subset if M CGIs can be selected as a range within the list of CGIs, such as the first M CGIs, or the second M CGIs, and so on through the last M CGIs.

As an example, N CGIs can be selected from the first M CGIs in TABLE A. It has been determined that a number N of about 300-400 selected from a number M of about 350-450 of the CGIs can be particularly useful, such as about 350 CGIs of the first 400 CGIs. Other example selections include but are not limited to about 300 CGIs of the second 400 CGIs, about 300 CGIs of the third 400 CGIs, about 300 CGIs of the last 400 CGIs, etc. Different combinations of different selections also can be made.

As another example, given a ranking of candidate CGIs, a top X % of the ranked CGIs can be selected, wherein X is a positive number. The top X % can be defined by a numerical threshold applied to the statistical metric used to rank the CGIs. For example, given the candidate CGIs in TABLE A, when ranked, the top 20% to 25% can be selected. As examples, the top 20% can be selected, the top 21% can be selected, the top 22% can be selected, the top 23% can be selected, the top 24% can be selected, or the top 25% can be selected, or a fractional percentage between these can be selected.

The foregoing description provides example implementations of a computer system implementing these techniques. The various computers used in this computer system can be implemented using one or more general-purpose computers, such as client devices including mobile devices and client computers, one or more server computers, or one or more database computers, or combinations of any two or more of these, which can be programmed to implement the functionality such as described in the example implementations.

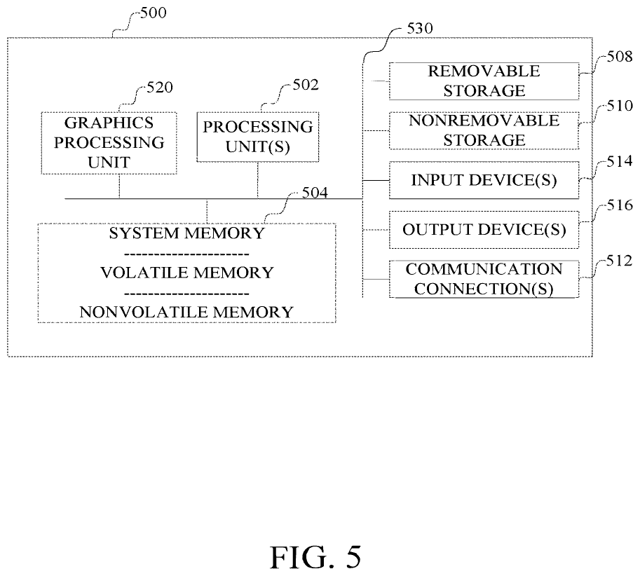

FIG. 5 is a block diagram of a general-purpose computer which processes computer programs using a processing system. Computer programs on a general-purpose computer generally include an operating system and applications. The operating system is a computer program running on the computer that manages access to resources of the computer by the applications and the operating system. The resources generally include memory, storage, communication interfaces, input devices and output devices.

Examples of such general-purpose computers include, but are not limited to, larger computer systems such as server computers, database computers, desktop computers, laptop and notebook computers, as well as mobile or handheld computing devices, such as a tablet computer, handheld computer, smart phone, media player, personal data assistant, audio and/or video recorder, or wearable computing device.

With reference to FIG. 5, an example computer 500 comprises a processing system including at least one processing unit 502 and a memory 504. The computer can have multiple processing units 502 and multiple devices implementing the memory 504. A processing unit 502 can include one or more processing cores (not shown) that operate independently of each other. Additional co-processing units, such as graphics processing unit 520, also can be present in the computer. The memory 504 may include volatile devices (such as dynamic random-access memory (DRAM) or other random-access memory device), and non-volatile devices (such as a read-only memory, flash memory, and the like) or some combination of the two, and optionally including any memory available in a processing device. Other memory such as dedicated memory or registers also can reside in a processing unit. Such a memory configures is delineated by the dashed line 504 in FIG. 5. The computer 500 may include additional storage (removable and/or non-removable) including, but not limited to, solid state devices, or magnetically recorded or optically recorded disks or tape. Such additional storage is illustrated in FIG. 5 by removable storage 508 and non-removable storage 510. The various components in FIG. 5 are generally interconnected by an interconnection mechanism, such as one or more buses 530.

A computer storage medium is any medium in which data can be stored in and retrieved from addressable physical storage locations by the computer. Computer storage media includes volatile and nonvolatile memory devices, and removable and non-removable storage devices. Memory 504, removable storage 508 and non-removable storage 510 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 500 may also include communications connection(s) 512 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals. Communications connections 512 are devices, such as a network interface or radio transmitter, that interface with the communication media to transmit data over and receive data from signals propagated through communication media.

The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 500 may have various input device(s) 514 such as a various pointer (whether single pointer or multi-pointer) devices, such as a mouse, tablet and pen, touchpad and other touch-based input devices, stylus, image input devices, such as still and motion cameras, audio input devices, such as a microphone. The compute may have various output device(s) 516 such as a display, speakers, printers, and so on, also may be included. These devices are well known in the art and need not be discussed at length here.

The various storage 510, communication connections 512, output devices 516 and input devices 514 can be integrated within a housing of the computer, or can be connected through various input/output interface devices on the computer, in which case the reference numbers 510, 512, 514 and 516 can indicate either the interface for connection to a device or the device itself as the case may be.

An operating system of the computer typically includes computer programs, commonly called drivers, which manage access to the various storage 510, communication connections 512, output devices 516 and input devices 514. Such access generally includes managing inputs from and outputs to these devices. In the case of communication connections, the operating system also may include one or more computer programs for implementing communication protocols used to communicate information between computers and devices through the communication connections 512.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system.

Each component (which also may be called a “module” or “engine” or “computational model” or the like), of a computer system such as described herein, and which operates on one or more computers, can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

Reference numbers in brackets “[ ]” herein refer to the corresponding literature listed in the attached Bibliography which forms a part of this Specification, and the literature is incorporated by reference herein.

The tables listed below are referred to in this Specification, form a part of this Specification, and are hereby incorporated by reference into this Specification:

Table A—Listing of CGIs (“TABLE A”).

It should be understood that the subject matter defined in the appended claims is not necessarily limited to the specific implementations described above. The specific implementations described above are disclosed as examples only.

What is claimed is:

1. A process for detecting a risk of presence of an early stage neoplasm in a subject based on a liquid biopsy sample from the subject, the liquid biopsy sample containing cell-free DNA fragments, the process comprising:
determining methylation information of the cell-free DNA fragments originating from cancer informative CGIs;
using an analytical platform, computing, for one or more of the cancer-informative CGIs, a respective plurality of metrics based on methylation information of respective located cell-free DNA fragments originating from the cancer-informative CGI, wherein the plurality of metrics comprises at least a transition metric reflecting a number of transitions between differentially methylated neighboring CpG sites;
processing the plurality of metrics for the cancer-informative CGIs with a computational model, the computational model providing an output indicating a likelihood of presence in the individual of an early stage neoplasm, wherein the computational model is based on a training set including samples containing the cancer informative CGIs originating from individuals known to have the early stage neoplasm.

2. The process of claim 1, further comprising detecting or monitoring, by the analytical platform, presence of early-stage cancer or a precancerous state or condition or to predict a likelihood of a cancer or a precancerous state or condition using the cell-free DNA fragments.

3. The process of claim 1, wherein the plurality of metrics comprises at least a proportion metric.

4. The process of claim 1, wherein the liquid biopsy sample comprises plasma obtained from an asymptomatic individual.

5. The process of claim 1, further comprising predicting, based on one or more metrics of the plurality of metrics and the cancer informative CGIs, a likelihood of presence of a neoplasm in the subject.

6. The process of claim 5, wherein the neoplasm is an early-stage cancerous solid tumor.

7. The process of claim 1, wherein the selected cancer informative CGIs include those having a statistical metric above a threshold based on a training set and one or more metrics computed for samples in the training set.

8. The process of claim 7, wherein the one or more metrics comprises at least a proportion metric.

9. The process of claim 1, wherein the computational model predicts the likelihood of presence of a precancerous neoplasm or early stage cancer in the subject with a sensitivity greater than a first threshold and a specificity greater than a second threshold.

10. The process of claim 9, the sensitivity is greater than about 70% at a specificity of greater than about 95%.

11. The process of claim 1, wherein the cancer informative CGIs are selected from a group consisting of a ranked set of candidate CGIs selected from TABLE A.

12. The process of claim 1, wherein the average number of cell-free DNA fragments processed per cancer informative CGI is greater than 200.

13. A machine for detecting a risk of presence of an early stage neoplasm in a subject based on a liquid biopsy sample from the subject, the liquid biopsy sample containing cell-free DNA fragments, the machine comprising:
computer storage which stores data representing methylation information of cell-free DNA fragments from the liquid biopsy sample and originating from cancer informative CG islands (CGIs)
a processing system accessing the computer storage to compute, for one or more cancer-informative CGI, a respective plurality of metrics based on the methylation information of the respective located cell-free DNA fragments originating from the cancer-informative CGI, and to store for the subject, for each of the cancer informative CGIs, the respective computed plurality of metrics for the cancer informative CGI, wherein the plurality of metrics comprises a transition metric reflecting a number of transitions between differentially methylated neighboring CpG sites; and
the processing system further processing the plurality of metrics for the cancer-informative CGIs with a computational model, the computational model providing an output indicating a likelihood of presence in the individual of an early stage neoplasm, wherein the computational model is based on a training set including samples containing the cancer informative CGIs originating from individuals known to have the early stage neoplasm.

14. The machine of claim 13, wherein the average number of cell-free DNA fragments processed per cancer informative CGI is greater than 200.

15. The machine of claim 13, wherein the plurality of metrics comprises at least a proportion metric.

16. The machine of claim 13, wherein the computational model is configured to predict a likelihood of presence of a neoplasm in the subject based on one or more metrics of the computed plurality of metrics and the cancer informative CGIs.

17. The machine of claim 16, wherein the neoplasm is an early-stage cancerous solid tumor.

18. The machine of claim 13, wherein the selected cancer informative CGIs include those having a statistical metric above a threshold based on a training set and one or more metrics computed for samples in the training set.

19. The process of claim 18, wherein the one or more metrics comprises at least a proportion metric.

20. The machine of claim 13, wherein the average number of cell-free DNA fragments processed per cancer informative CGI is greater than 200.
